SEQUENCE LISTING (14 sequences)

SEQ ID NO: 1 - DNA, 24 bp, Artificial Sequence
  VEGF sense primer
tttctgctgtcttgggtgcattgg

SEQ ID NO: 2 - DNA, 24 bp, Artificial Sequence
  VEGFa antisense primer
accacttcgtgatgattctgccct

SEQ ID NO: 3 - DNA, 24 bp, Artificial Sequence
  TSP 1 sense primer
ttccgccgattccagatgattcct

SEQ ID NO: 4 - DNA, 24 bp, Artificial Sequence
  TSP 1 antisense primer
acgagttctttaccctgatggcgt

SEQ ID NO: 5 - DNA, 24 bp, Artificial Sequence
  HIFIa sense primer
ttggcagcaacgacacagaaactg

SEQ ID NO: 6 - DNA, 24 bp, Artificial Sequence
  HIFIa antisense primer
ttgagtgcagggtcagcactactt

SEQ ID NO: 7 - DNA, 24 bp, Artificial Sequence
  GAPDH sense primer
agcctcaagatcatcagcaatgcc

SEQ ID NO: 8 - DNA, 24 bp, Artificial Sequence
  GAPDH antisense primer
tgtggtcatgagtccttccacgat

SEQ ID NO: 9 - DNA, 981 bp, Homo sapiens
  CDS (1)..(981)
  ref <308>: DATABASE ACCESSION NUMBER: NM_001419.2
  ref <309>: DATABASE ENTRY DATE: 2010-09-19
  ref <313>: RELEVANT RESIDUES IN SEQ ID NO: (1)..(981)
atgtctaatggttatgaagaccacatggccgaagactgcaggggtgacatcgggagaacgaatttgatcgtcaactacctccctcagaacatgacccaggatgagttacgaagcctgttcagcagcattggtgaagttgaatctgcaaaacttattcgggataaagtagcaggacacagcttgggctatggctttgtgaactacgtgaccgcgaaggatgcagagagagcgatcaacacgctgaacggcttgaggctccagtcaaaaaccattaaggtgtcgtatgctcgcccgagctcagaggtgatcaaagacgccaacttgtacatcagcgggctcccgcggaccatgacccagaaggacgtagaagacatgttctctcggtttgggcggatcatcaactcgcgggtcctcgtggatcagactacaggtttgtccagaggggttgcgtttatccggtttgacaaacggtcggaggcagaagaggcaattaccagtttcaatggtcataaacccccaggttcctctgagcccatcacagtgaagtttgcagccaaccccaaccagaacaaaaacgtggcactcctctcgcagctgtaccactcgccagcgcgacggttcggaggccccgttcaccaccaggcgcagagattcaggttctcccccatgggcgtcgatcacatgagcgggctctctggcgtcaacgtgccaggaaacgcctcctccggctggtgcattttcatctacaacctggggcaggatgccgacgaggggatcctctggcagatgtttgggccgtttggtgccgtcaccaatgtgaaagtgatccgcgacttcaacaccaacaagtgcaaagggtttggctttgtgaccatgacaaactatgaagaagccgcgatggccatagccagcctgaacggctaccgcctgggggacaaaatcttacaggtttccttcaaaaccaacaagtcccacaaataa

SEQ ID NO: 10 - protein, 326 aa, Homo sapiens
MSNGYEDHMAEDCRGDIGRTNLIVNYLPQNMTQDELRSLFSSIGEVESAKLIRDKVAGHSLGYGFVNYVTAKDAERAINTLNGLRLQSKTIKVSYARPSSEVIKDANLYISGLPRTMTQKDVEDMFSRFGRIINSRVLVDQTTGLSRGVAFIRFDKRSEAEEAITSFNGHKPPGSSEPITVKFAANPNQNKNVALLSQLYHSPARRFGGPVHHQAQRFRFSPMGVDHMSGLSGVNVPGNASSGWCIFIYNLGQDADEGILWQMFGPFGAVTNVKVIRDFNTNKCKGFGFVTMTNYEEAAMAIASLNGYRLGDKILQVSFKTNKSHK

SEQ ID NO: 11 - DNA, 58 bp, Artificial Sequence
  Sense-strand TOPO primer encoding HA tag and 5′ end of human HuR gene
caccatgtacccatacgatgttccagattacgctcttatgtctaatggttatgaagac

SEQ ID NO: 12 - DNA, 21 bp, Artificial Sequence
  Anti-sense TOPO primer anchored at the 3′ end of the human HuR gene
ttatttgtgggacttgttggt

SEQ ID NO: 13 - DNA, 33 bp, Artificial Sequence
  Artificial sequence encoding an HA tag
atgtacccatacgatgttccagattacgctctt

SEQ ID NO: 14 - DNA, 18 bp, Artificial Sequence
  Artificial sequence encoding shRNA H760
ggatcctctggcagatgt

(19) United States
(12) Patent Application Publication
(10) Pub. No.: US 2011/0110896 A1
(43) Pub. Date: May 12, 2011

(54) Modulating levels of RNA-binding proteins for the treatment of breast cancer

(75) Inventors: Ulus Atasoy, Columbia, MO (US); Matthew Michael Gubin, Columbia, MO (US); Robert Calaluce, Mexico, MO (US)

(73) Assignee: The Curators of the University of Missouri, Columbia, MO (US)

(21) Appl. No.: 12/924,953

(22) Filed: Oct. 8, 2010

Related U.S. Application Data
(60) Provisional application No. 61/278,761, filed on Oct. 9, 2009.

(51) Int. Cl. A61K 31/713; A61K 31/7088; A61K 35/76; A61P 35/00; A61P 35/04; C12N 5/09; C07H 21/00; C07H 21/04; C12N 15/63

(52) U.S. Cl. 424/93.6; 514/44 R; 514/44 A; 435/375; 536/23.5; 435/320.1; 536/24.5

(57) ABSTRACT

The present invention relates to methods of using RNA-binding protein modulating agents to treat of cancer, particularly patients that are susceptible to or diagnosed with estrogen receptor-negative breast cancer, such as methods of inhibiting the growth or metastasis of cancer cells comprising contacting cells with a therapeutically-effective amount of an HuR-modulating agent. The invention also relates to compositions comprising therapeutically-effective amounts of an HuR-modulating agent.

### CROSS-REFERENCE TO RELATED APPLICATIONS

The pending application claims priority claims under 35 U.S.C. §119(e) to U.S. Provisional Patent Application No. 61/251,921, filed Oct. 9, 2009, the disclosure of which is incorporated herein by reference.

### STATEMENT REGARDING FEDERALLY-SPONSORED RESEARCH OR DEVELOPMENT

This invention was made with government support under the contract No. W81XWH07-1-0406 awarded by the ARMY/MRMC under the Department of Defense. The government has certain rights in the invention.

### INCORPORATION BY REFERENCE OF MATERIAL SUBMITTED ON A COMPACT DISK

The sequence listing contained in the file “UMCO-D686U1 (10UMC005)_ST25.txt” recorded on 2010-10-08 is incorporated by reference in its entirety herein.

### FIELD OF THE INVENTION

The present invention relates to methods of using RNA-binding protein modulating agents to treat cancer, particularly patients that are susceptible to or diagnosed with estrogen receptor-negative breast cancer, such as methods of inhibiting the growth or metastasis of cancer cells by contacting cells with a therapeutically-effective amount of an HuR-modulating agent. The invention also relates to compositions comprising therapeutically-effective amounts of an HuR-modulating agent.

### BACKGROUND OF THE INVENTION

Post-transcriptional gene regulation, mediated by RNA-binding proteins (RBPs) and microRNAs (miRNAs), is now recognized as playing an important role in the development of cancerous cells (Deng S, et al., Cell Cycle 2008; 7:2643-6; Esquela-Kerscher A and Slack F J., Nat Rev Cancer 2006; 6:259-69; Keene J D., Proc Natl Acad Sci USA 1999; 96:5-7; Keene J D., Proc Natl Acad Sci USA 2001; 98:7018-24; Keene J D., Mol Cell 2003; 12:1347-9; Keene J D., Nat Genet 2003; 33:111-2; Keene J D and Tenenbaum S A. Mol Cell 2002; 9:1161-7). While a variety of techniques have been used to identify and study the transcription of many cancer-related genes, many traditional methods, such as microarray profiling, do not detect changes in the levels of transcripts of genes that are unaltered in different cellular states. Methods involving immunoprecipitation of RNAs applied to microarrays (RIP-Chips), however, have greatly facilitated the identification and study of unique mRNAs overlooked by traditional methods (Calaluce et al., BMC Cancer 2010, 10:126).

The discovery of post-transcriptional gene regulation has stimulated interest in identifying gene products involved in the acquired capabilities model of malignant transformation (Hanahan, D, and Weinberg R A, Cell 2000; 100:57-70). HuR, an RBP that is overexpressed in many malignant cells, is recognized as a paraneoplastic antigen that may function as a tumor maintenance gene (Gorospe M., Cell Cycle 2003; 2:412-4; Abdelmohsen K, et al., Cell Cycle 2010; 9; Atasoy U, et al., J Cell Sci 1998; 111:3145-56; Dalmau J, et al., Ann Neurol 1990; 27:544-52; Dalmau J, et al., Neurology 1991; 41:1757-64; Fan X C and Steitz J A. EMBO J 1998; 17:3448-60; Ma W J, et al., J Biol Chem 1996; 271:8144-51; Lopez de Silanes I, et al., RNA Biol 2005; 2:11-3). HuR regulates genes in many areas of the acquired capabilities model, including two genes known to play an important role in the regulation of angiogenesis, VEGF and HIF1α (Abdelmohsen K, et al, Cell Cycle 2007; 6:1288-92; Goldberg-Cohen I, et al, J Biol Chem 2002; 277:13635-40; Levy A P., Trends Cardiovasc Med 1998; 8:246-50; Levy N S, et al, J Biol Chem 1998; 273:6417-23; Galban S, et al., Mol Cell Biol 2008; 28:93-107). Increased cytoplasmic expression of HuR is directly correlated with severity and aggressiveness of many cancers, including human breast cancer (Heinonen M, et al. Cancer Res 2005; 65:2157-61; Heinonen M, et al. Clin Cancer Res 2007; 13:6959-63).

Breast cancer is broadly divided into two different subtypes: estrogen receptor positive (ER+) and estrogen receptor negative (ER−). The majority of women with breast cancer are ER+(85%), and the remainder is ER− (15%) (Reis-Filho J S and Tutt A N., Histopathology 2008; 52:108-18). Patients with ER+breast cancer can be treated with the tamoxifen, but many of them develop drug resistance for unknown reasons (Hostetter C, et al. Cancer Biol Ther 2008; 7). The prognosis for women with ER− breast cancer, which disproportionately affects lower income and minority women, is poor, with dismal survival rates. There are no specific treatments for women with ER− breast cancer. These patients are often treated with surgery and chemotherapy, but the cancer eventually recurs, resulting in death. Therefore, there is a need to develop novel therapies to treat breast cancer, particularly patients having ER− breast cancer, and therapies designed to overcome the development of resistance to tamoxifen in ER+ breast cancer patients.

### SUMMARY OF THE INVENTION

The invention relates to a method of inhibiting the growth or metastasis of cancer cells comprising contacting cells with a therapeutically-effective amount of an HuR-modulating agent. The invention also relates to a composition comprising a therapeutically-effective amount of an HuR modulating agent capable of inhibiting the growth or metastasis of cancer cells. The compositions and methods can be used to treat women who are susceptible to or diagnosed with hormone receptor (estrogen or progesterone receptor) negative (ER−) breast cancer.

### BRIEF DESCRIPTION OF THE DRAWINGS

The foregoing aspects, features, and advantages of the invention, as well as presently preferred embodiments thereof, will become more apparent from the drawings and the detailed description, set forth below.

FIG. 1 sets forth illustrations demonstrating that overexpression of HA HuR in MDA-MB-231 cancer cells increases cell growth and alters cell cycle kinetics in vitro while inhibiting tumor growth in vivo. (A) MDA-MB-231 cells transfected with the pZeoSV2 vector expressing HA HuR, selected with Zeocin, and cloned by limiting dilution, express HA HuR compared to a pZeoSV2 empty vector control. MDA-MB-231 clones 4E1 and 5F1 expressed 42% and 38% more HuR than the control. (B) Both clones expressing HA HuR proliferated significantly faster than the empty vector control in vitro. (C) Overexpression of HA HuR increased cells in G₀/G₁ cell cycle, from 57.70% to 67.67%. Overexpression of HA HuR also decreased cells in G₂/M phase by 27.19% to 18.29%, but did not significantly alter cells in S phase as measured by DNA content. (D) MDA-MB-231 HA HuR 4E1 showed significantly reduced tumor volume (mm³) and growth starting at two weeks post-inoculation, and continuing for five weeks, compared to empty vector MDA-MB-231 controls measured by both MRI and calipers. For tumor experiments, nine animals per group were used. Counting assay and tumor volume data values are represented as mean value±SEM. p<005. Flow cytometry data are represented as mean value±SEM from n=4 separate experiments done in triplicate. p<005.

FIG. 2 sets forth an illustration demonstrating that RT-PCR confirms the presence of HA HuR mRNA in the left tumor. (A) RT-PCR using primers specific for HA HuR confirms that HA HuR mRNA is expressed in the left tumor, compared to the right tumor empty vector control. DNA was resolved on 1% agarose gels and visualized by ethidium bromide staining. GAPDH was used as an internal control. pZeo SV2 HA HuR plasmid and distilled water (DNAse- and RNAse-free) served as positive and negative controls, (n=1 representative set of tumors). (B) HA-HuR protein is expressed in the left (HA-HuR tumor) but not the right (EV control tumor) as determined by western blot. (n=2 sets of tumors).

FIG. 3. sets forth an illustration demonstrating that cells in culture re-derived from tumors over-expressing HA HuR proliferate faster than empty vector control in vitro. Left and right tumor cells extracted from tumors over-expressing HuR (left) grown in culture proliferate significantly faster than control tumors expressing an empty vector (right), (n=1, representative set of tumors).

FIG. 4 sets forth illustrations demonstrating overexpression of HA HuR in MDA-MB-231 cancer cells inhibits tumor growth in athymic nude mice. (A) Repeat experiments comparing MDA-MB-231 HA HuR 4E1 with both wild-type MDA-MB-231 and empty vector MDA-MB-231 confirmed that HuR overexpression reduced tumor volume (mm³) and growth starting at two weeks post-inoculation, and continuing for five weeks, as measured by calipers. (B) Tumors overexpressing HA HuR had significantly less mass after harvest 42 days post-inoculation compared to the WT or empty vector (EV) controls. (C) Magnetic Resonance Imaging comparing largest MRI sections for each tumor showed significantly smaller tumors in the HA HuR overexpressing tumors compared to EV control tumors. (D) UPPER: Representative cross sections of tumors showed that those formed by inoculation with HA HuR resembled a gelatin capsule, were significantly smaller, and more homogeneous than those formed by inoculation with the empty vector. LOWER: Hematoxylin and eosin stain revealed poorly differentiated carcinomas having similar morphology and lacking inflammatory cells in both HA HuR tumors (F) and EV control tumors (G). Five animals were used in HA HuR, empty vector, and wild-type control groups. When experiments were repeated using a different clone, similar results were obtained (see FIG. 3). Data represent mean value±SEM. p<0.0005.

FIG. 5 sets forth an illustration demonstrating that MDA-MB-231 cells over-expressing HuR clone 5F1 show significantly reduced tumor growth. (A) MDA-MB-231 cells overexpressing HuR (clone 5F1) had reduced volume compared to the empty vector control starting at day 14 and continuing through day 35. (B) MDA-MB-231 cells over-expressing HuR (clone 5F1) had significantly reduced tumor mass compared to the empty vector control. Data represent mean value±SEM. n=5 mice. p<0.05.

FIG. 6 sets forth illustrations demonstrating Gene Ontology (GO) analysis. (A and B) Gene Ontology analysis revealed differentially expressed genes in the HA-HuR tumors compared to the empty vector (EV) control tumors, which are more represented in the Biological Processes (BP) or Molecular Function (MF) GO categories than expected due to chance.

FIG. 7 sets forth illustrations demonstrating that TSP1 is up-regulated in HA HuR tumor while VEGFa is down-regulated. (A) Real-time PCR indicates TSP1 is up-regulated in tumors (5.44-fold) and cells in culture (4.88-fold) overexpressing HA HuR compared to EV control tumors and cells, which are consistent with the microarray data. VEGF is down-regulated (3.2- and 2.6-fold, respectively) in tumors overexpressing HA HuR compared to EV controls. HIF1a mRNA levels did not appreciably change. The change in gene expression was determined using the comparative CT method and is represented as the fold change in HA HuR tumors compared to empty vector controls. GAPDH was used as an endogenous control. (B) Western blot for TSP1 shows increased protein expression of TSP1 (76%) in the HA HuR over-expressing tumors compared to EV control tumors. (C) Western blot for VEGF shows decreased protein expression by 23% in the HA HuR overexpressing tumors compared to EV control tumors (representative of two independent sets of tumors). Data represent mean value±SEM from n=3 separate mice done in triplicate. p<0.005.

FIG. 8 sets forth illustrations demonstrating that tumors over-expressing HA-HuR have no increases in apoptosis but decreased blood vessel formation compared to control. (A) Annexin V staining reveals similar amounts of cells undergoing apoptosis as compared to cells overexpressing HA-HuR to EV control cells. (B) Caspase 3 staining shows no differences in the amount of apoptosis in the EV control tumors compared to HA-HuR tumors harvested 14 days post-inoculation. In the tumors harvested on day 42 post-inoculation, caspase 3 staining showed more apoptotic cells in the EV control tumors compared to tumors overexpressing HA-HuR. CD34 staining shows fewer blood vessels in tumors over expressing HA-HuR. (C) Quantitation of blood vessels stained (number of vessels per high power field scored) with CD34 indicate significantly fewer blood vessels in the tumors over-expressing HA-HuR. n=3 pairs of tumors from separate mice. Error bars ±SEM; p<0.005; in photomicrographs bar=27 microns. Representative of n=5 sets of tumors.

FIG. 9 sets forth an illustration demonstrating that a senescence assay reveals reduced staining in the tumor overexpressing HuR. Senescence staining for β-galactosidase showed fewer senescent cells in the tumor that was overexpressing HuR compared to the empty vector control. In the photomicrographs, the bar represents 27 microns. Representative of n=5 sets of tumors.

FIG. 10 sets forth illustrations demonstrating HuR expression in MB-231 cells. (LEFT) MB-231 cells over expressing HuR have significantly reduced levels of SOX4 and CXCR4 mRNA. MB-231 cells overexpressing HuR (clone 4E1) have approximately a 75-fold reduction in SOX4 mRNA and a 17-fold reduction in CXCR4 mRNA when compared to empty vector control (clone 2C7). n=2 in triplicate. p<0.05. (RIGHT) MB-231 tumors over expressing HuR have significantly reduced levels of SOX4 and CXCR4 mRNA MB-231 tumors over expressing HuR (clone 4EI) have approximately a 32-fold reduction in SOX4 mRNA and a 22-fold reduction in CXCR4 mRNA compared to the empty vector control (clone 2C7). n=2 in triplicate. p<0.05.

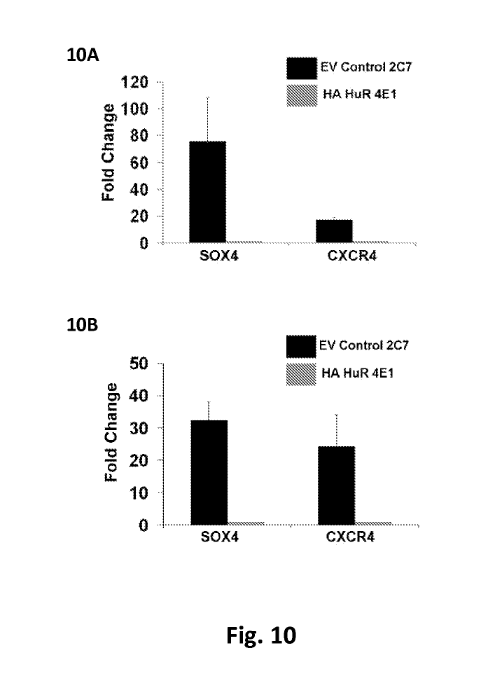

FIG. 11 sets forth illustrations demonstrating that HuR interacts with both TSP1 and VEGF mRNAs in cells overexpressing HuR. (A) RNA immunoprecipitation indicates both TSP1 and VEGF mRNA are increased in the HuR IP when compared to IgG1 control IP in both HA-HuR overexpressing cells and EV control cells. (B) Actinomycin D. mRNA stability assay shows VEGF mRNA half-life was not altered between cells overexpressing HA-HuR and EV control cells. (C) TSP1 mRNA from cells overexpressing HA-HuR has a longer half-life than TSP1 mRNA from EV control cells. For RNA immunoprecipitation, data represents mean value±SEM. p<0.05.

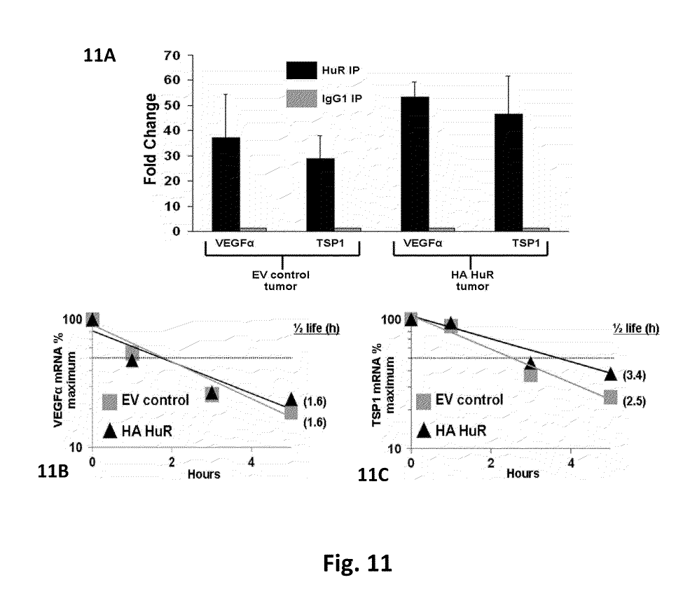

FIG. 12 sets forth an illustration demonstrating that apoptosis is not altered in vitro between HA-HuR overexpressing cells compared to EV control cells. 7-AAD staining revealed a similar number of cells undergoing apoptosis or necrosis (7-AAD intermediate or 7-AAD high) when HA-HuR overexpressing cells and EV control cells were compared. Data represent mean value±SEM. n=3.

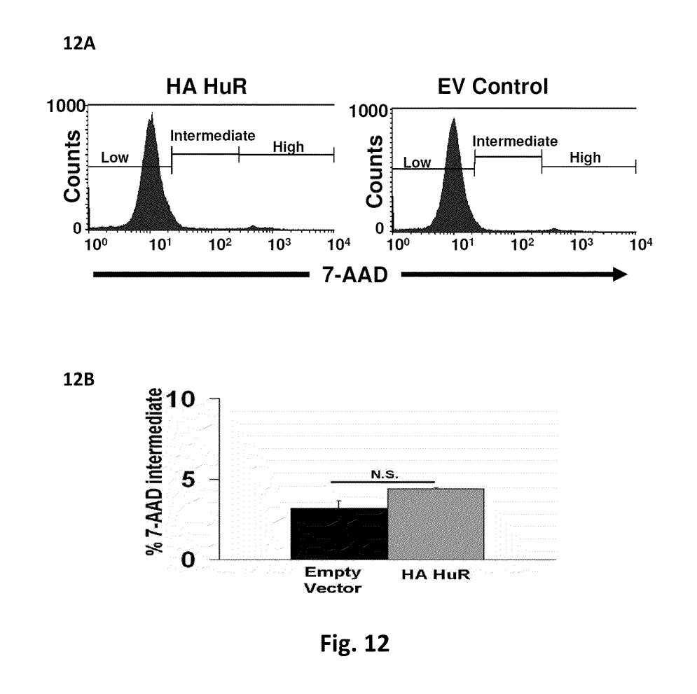

FIG. 13 sets forth an illustration demonstrating that TUNEL staining confirmed that alterations in apoptosis was not observed in tumors that overexpress HA-HuR and in EV control tumors at day 14 post-inoculation. TUNEL staining indicated HA-HuR tumors harvested on day 42, however, had fewer cells undergoing apoptosis compared to EV control tumors. Quantitation of apoptosis (number of apoptotic cells per high power field scored) showed that alterations in apoptosis was not observed the HA-HuR tumor cells and in the EV control tumors at day 14. Data represent mean value ±SEM. Representative of n=5 sets of tumors. p<0.05.

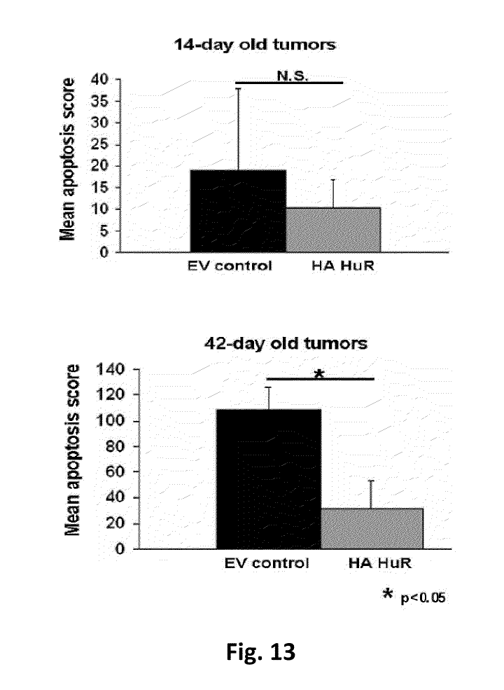

FIG. 14 sets forth an illustration demonstrating hematoxylin and eosin staining of tumors. Tumors harvested on day 14 show morphology consistent with adenocarcinoma in both HA-HuR overexpressing tumors and EV control tumors.

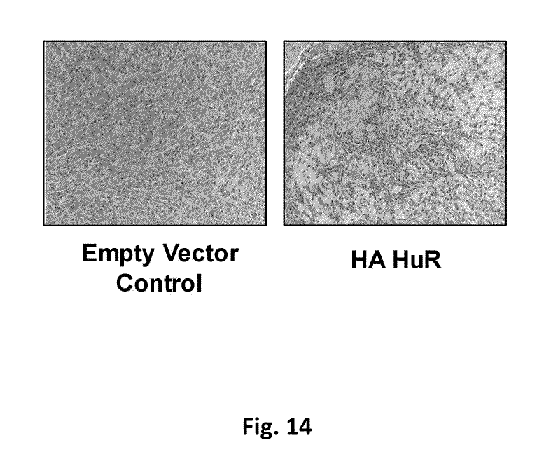

FIG. 15 sets forth an illustration showing the genetic elements and cloning site of the pLenti 7.3/V5 TOPO® cloning vector. The genetic elements of the vector are show in Panel A and the nucleotide sequence flanking the TOPO cloning site are shown in Panel B.

FIG. 16 sets forth an illustration showing how the ViraPower packaging mix is used to prepare recombinant lentiviral particles are prepared using the ViraPower™ Packaging mix and introduced into mammalian cells to express a desired recombinant protein.

FIG. 17 sets forth an illustration demonstrating that over-expression of HuR with a lentivirus in MDA-MB-231 cells significantly inhibits tumor growth. MDA-MB-231 cells infected with a lentivirus overexpressing HA HuR showed significantly reduced tumor volume (mm³) and growth starting at five weeks post-inoculation and continuing for fourteen weeks when compared to MDA-MB-231 infected with a lentivirus expressing LacZ control. Five animals per group were used. p<0.05.

FIG. 18 illustrates a circular arrangement of genetic elements of the pLentiLox 3.7 expression vector used to clone and express an shRNA directed against HuR. The nucleotide sequence encoding the shRNA designated H760 was cloned into the HpaI and XhoI sites of the pLentiLox 3.7 backbone viral plasmid downstream of the U6 promoter.

FIG. 19 illustrates a linear arrangement of genetic elements in the LentiLox-shRNA H760 expression vector. The nucleotide sequence encoding shRNA H760 was cloned in the multiple cloning site (MCS) region downstream of the U6 promoter. The gene encoding a variant of the Green Fluorescent Protein (EGFP) is located downstream from the CMV promoter.

FIG. 20 sets forth an illustration demonstrating that under-expression of HuR with a lentivirus expressing a shRNA targeting HuR in MDA-MB-231 cells significantly inhibits tumor growth. MDA-MB-231 cells infected with a lentivirus expressing a shRNA knocking down HuR (LL HuR shRNA) showed significantly reduced tumor volume (mm³) and growth starting at seven weeks post-inoculation and continuing for fourteen weeks when compared to MDA-MB-231 infected with a lentivirus expressing no shRNA (LL control). Five animals per group were used. p<0.05.

Unless otherwise defined, all technical and scientific terms used herein have the same meaning as commonly understood by one of ordinary skill in the art to which this invention belongs. Abbreviations and their corresponding meanings include: aa or AA=amino acid; ER=estrogen receptor; mg=milligram(s); ml or mL=milliliter(s); mm=millimeter(s); mM=millimolar; nmol=nanomole(s); ORF=open reading frame; PCR=polymerase chain reaction; pmol=picomole(s); ppm=parts per million; RT=reverse transcriptase; RT=room temperature; SDS-PAGE=sodium dodecyl sulfate-polyacrylamide gel electrophoresis; U=units; ug, pg=micro gram(s); ul, μl=microliter(s); and uM, μM micromolar; Estrogen receptor negative (ER−), estrogen receptor positive (ER+), RNA immunoprecipitation (RIP), RNA immunoprecipitation applied to microarrays (RIP-Chip), 3′ untranslated region (3′ UTR), ELAV1 (embryonic lethal abnormal vision 1).

### DETAILED DESCRIPTION OF THE INVENTION

The following detailed description illustrates the invention by way of example and not by way of limitation. The description clearly enables one skilled in the art to make and use the invention, describes several embodiments, adaptations, variations, alternatives, and uses of the invention, including what is presently believed to be the best mode of carrying out the invention.

The present invention relates to new and improved therapies to treat cancer, particularly cancer mediated by ER− breast cancer cells, involving methods of modulating the expression of HuR in cancer cells. The studies of HuR expression in ER− breast cancer cells described below, demonstrate that over-expression of epitope tagged (HA) HuR in MDA-MB-231 resulted in cell lines that have higher cell growth rates and alterations in their cell cycle kinetics. When these are used in xenograft models of cancer (athymic mice), a 90% reduction in growth rates was observed, compared to control groups. These results suggest that HuR may have a direct or indirect antiangiogenic affect by increasing the expression of thrombospondin 1 (TSP1), a gene known to have anti-angiogenic properties. We also observed increases in steady state mRNA levels of thrombospondin 2 (TSP2), another potent inhibitor of angiogenesis. VEGF expression was decreased, strongly suggesting that that overexpression of HuR in ER− breast cancer cells can inhibit tumor growth by blocking angiogenesis. MB-231 cells that overexpress HuR also have significantly reduced levels of SOX4 and CXCR4 mRNAs, and that MB-231 tumors that overexpress HuR have significantly reduced levels of SOX4 and CXCR4 mRNAs. These observations support the idea that methods which modulate the level of HuR in cancer cells, particularly overexpression of HuR in ER− breast cancer cells, will reduce the rate of metastasis by these and similar types of cells.

The invention relates to a method of inhibiting the replication or metastasis of cancer cells comprising contacting cells with a therapeutically-effective amount of an HuR-modulating agent. In a preferred aspect of the invention, the HuR-modulating agent increases or decreases the level of expression of the RNA-binding protein HuR by more than three-fold in a sample comprising cancer cells contacted with the HuR-modulating agent compared to control sample of cancer cells not contacted with the HuR-modulating agent. In a more preferred aspect of the invention, the level of expression of HuR is increased. In a more preferred aspect of the invention, the level of expression of HuR is decreased.

A variety of methods may be used to alter the level of HuR in cancer cells. A gene encoding HuR operably-linked to a promoter, for example, can be cloned into a plasmid or a viral vector, which may be introduced into cells using standard transformation/transduction, or transfection techniques, respectively. The encoded HuR may also comprise an epitope tag (e.g., hemagglutinin, HA) that facilitates detection of the heterologous, tagged HuR from untagged native HuR present in the transformed or transfected cells. Lentiviral vectors, for example, can be used to transfect genes encoding tagged and un-tagged HuR into cancer cells. Double-stranded DNAs (dsDNAs, which may be linear, or circular, as in plasmids) covalently-linked to gold (Au) nanoparticles, for example, can also be used to introduce HuR gene constructs into cells by a variety of transformation methods, including particle bombardment methods.

In a preferred aspect of the invention, the effect of modulating HuR treats an HuR-mediated disease. In a more preferred aspect of the invention, the HuR-mediated disease is cancer. In an even more preferred aspect of the invention, the cancer is breast cancer, and in a most preferred aspect of the invention, the cancer cells are estrogen receptor negative breast cancer cells.

In a preferred aspect of the invention, the HuR-modulating agent comprises a single- or double-stranded nucleic acid comprising an HuR gene, or fragment thereof, operably-linked to a promoter active in cancer cells. In a preferred aspect of the invention, the nucleic acid is single-stranded.

In a more preferred aspect of the invention, the nucleic acid is single-stranded. In a more preferred aspect of the invention, the single-stranded nucleic acid is RNA. In an even more preferred aspect of the invention, the single-stranded RNA is one or more viral RNAs, which may be packaged in as a virus. In a most preferred aspect of the invention, the single-stranded RNA virus is a retrovirus. In a most preferred aspect of the invention, the retrovirus is a lentivirus.

Primary tumors of wild-type breast cancer cells (such as MDA-MB-231 or MCF-7) may be established in female athymic nude mice in mammary fat pads. Mice harboring MCF-7 cells may also be supplemented with estradiol pellets. HA-HuR may be cloned into a lentiviral backbone and then packaged into a VSV pseudo-typed lentivirus, which has GFP as a screening marker. HuR lentiviral stocks are prepared and titered, and then used to introduce different amounts of virus into a mouse by direct injection into a primary mammary tumor, or administered to a mouse systemically by intraperitoneal (i.p.) or intravenous (i.v.) injections. The efficiency of the injection may be assessed by using the Xenogen System (IVIS® 200 series pre-clinical imaging system, Caliper Life Sciences, Hopkinton, Mass.) Viruses harboring other marker genes, such as a lacZ gene encoding β-galactosidase, may be used as appropriate controls. One or more injections may be administrated, which may be repeated at different intervals, at the same, higher, or lower doses, depending upon the efficacy of the initial dosing schemes. The treatments (either intratumoral or systemic) may also be given at different times after initiation of tumor growth.

In a more preferred aspect of the invention, the nucleic acid is double-stranded. In a more preferred aspect of the invention, the double-stranded nucleic acid is DNA. In an even more preferred aspect of the invention, the double-stranded DNA is linear. In a most preferred aspect of the invention, the linear double-stranded DNA is a virus. The linear double-stranded viral DNA may also be packaged in a virus. In an even more preferred aspect of the invention, the double-stranded DNA is circular. The circular dsDNA can be a plasmid or a virus. The circular double-stranded viral DNA may also be packaged in a virus.

Double-stranded DNAs encoding HA-HuR may be covalently linked to covalently-linked nanoparticles may also be used to alter HuR expression in cancer cells. Gold (Au) nanoparticles, in different shapes (spherical or rod-shaped particles) or sizes (varying in diameter and length), may also be used. The nanoparticles may also contain one or more targeting molecules which recognize specific receptors on breast cancer cells, such as the bombesin peptide which shows high affinity for as Gastrin Releasing Peptide (GRP) receptor. Gold particles may be introduced into mice by intratumoral injection or systemic (i.p/i.v) delivery methods. The efficiency of transduction may be monitored by CT scans.

In a preferred aspect of the invention, the HuR gene, or fragment thereof, encodes an HuR polypeptide, or a fragment or variant thereof, capable of binding to mRNAs encoded by one or more genes involved in angiogenesis or metastasis. In a more preferred aspect of the invention, the level of expression of the HuR polypeptide, or a fragment of variant thereof, is increased in the cancer cells.

In a preferred aspect of the invention, the HuR gene, or fragment thereof, is operably-linked to the promoter active in cancer cells in an anti-sense direction. In a more preferred aspect of the invention, the level of expression of HuR is decreased in the cancer cells. The expression may be decreased, for example, using a nucleotide sequence encoding an shRNA, exemplified by shRNA H760, as shown in Example 3.

Testing animals are observed, and tumor growth is monitored weekly by caliper measurements, MRI scans, and when appropriate, Xenogen scans to detect expression of GFP marker proteins. PET scans may be performed on live animals to observe metastasis to distant organs. Different organs such as brain, lungs and bone marrow may also be assessed for metastasis after study animals are sacrificed. Primary tumors are weighed and analyzed by immunohistochemistry for relevant markers, such as HuR (HA-tagged and wild-type), VEGF, HIF1α, TSP1, and TSP2, and also for evidence of cellular apoptosis.

The invention also relates to a composition for inhibiting the replication or metastasis of cancer cells comprising a therapeutically-effective amount of an HuR modulating agent.

In a preferred aspect of the invention, the HuR-modulating agent increases or decreases the level of expression of the RNA-binding protein HuR by more than three-fold in a sample of cancer cells contacted with the HuR-modulating agent compared to control sample of cancer cells not contacted with the HuR-modulating agent. In one aspect of the invention the level of expression of HuR is increased. In another aspect of the invention it is decreased.

In a preferred aspect of the invention, administration of the modulating agent treats an HuR-mediated disease. In a more preferred aspect of the invention, the HuR-mediated disease is cancer, including breast cancer. In a more preferred aspect of the invention, the cancer cells are estrogen-receptor negative breast cancer cells.

In one aspect of the invention, the composition comprising the HuR-modulating agent comprises a single- or double-stranded nucleic acid comprising an HuR gene, or fragment thereof, operably-linked to a promoter active in cancer cells. In other aspects of the invention, the nucleic acid is single stranded, including single-stranded RNA, which may be packaged in a virus. Exemplary viruses include retroviruses, such as lentiviruses. In another aspect of the invention, the nucleic acid is double-stranded, such as double-stranded DNA. The composition may comprise linear double-stranded DNA, including linear dsDNAs that may be packaged in a virus. The composition may also comprise circular dsDNAs, such as in plasmid form, or as circular dsDNA packaged in a virus.

In a preferred aspect of the invention the HuR modulating agent comprises an HuR gene, or fragment thereof, which encodes an HuR polypeptide, or a fragment or variant thereof, capable of binding to mRNAs encoded by one or more genes involved in angiogenesis or metastasis. In one aspect of the invention, administration of the composition comprising the HuR modulating agent increases the level of expression of the HuR polypeptide, or a fragment of variant thereof, in the cancer cells. In an alternative aspect of the invention, the HuR modulating agent comprises an HuR gene, or fragment thereof, operably-linked to the promoter active in cancer cells in an anti-sense direction. In a preferred aspect of the invention, administration of this type of HuR modulating agent decreases the level of expression of HuR is decreased in the cancer cells. The expression may be decreased, for example, using a modulating comprising a nucleotide sequence encoding an shRNA, exemplified by shRNA H760, as shown in Example 3.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the appended claims and any equivalent thereof.

### EXAMPLES

The foregoing discussion may be better understood in connection with the following representative examples which are presented for purposes of illustrating the principle methods and compositions of the invention and not by way of limitation. Various other examples will be apparent to the person skilled in the art after reading the present disclosure without departing from the spirit and scope of the invention. It is intended that all such other examples be included within the scope of the appended claims.

### Example 1

### Materials and Methods

All parts are by weight (e.g., % w/w), and temperatures are in degrees centigrade (° C.), unless otherwise indicated.

Cell line and growth conditions. The MDA-MB-231 cell line was purchased from American Type Culture Collection (Manassas, Va.) and maintained at 37° C. in a humidified atmosphere of 95% air and 5% C0₂. The cells were grown in RPMI (GIBCO®, Invitrogen™, Carlsbad, Calif.), supplemented with 10% fetal calf serum (Hyclone, Thermo Fisher Scientific, Waltham, Mass.), 0.5 mM L-glutamine (GIBCO®), 25 mg/ml glucose (Sigma-Aldrich, St. Louis, Mo.), HEPES (GIBCO®) and Sodium Pyruvate (GIBCO®).

Generation of clones expressing HA HuR. Hemagglutinin (HA)-tagged human HuR was cloned into the NheI and XhoI sites of the pZeoSV2 (−) (Invitrogen™) vector. Cells were plated and then transfected with either pZeo HA HuR or pZeo empty vector using Lipofectamine 2000 (Invitrogen™). Five days after transfection, the media was removed and replaced with fresh medium containing 200 _82 g/ml of Zeocin antibiotic (Invitrogen™). Cells were selected for a ten day period. After ten days, selected cells were maintained in 50 μg/ml of Zeocin to maintain pZeo HA HuR and empty vector expression. No viable cells remained in the untransfected well. Cells were then cloned by limiting dilution.

SDS-PAGE and Western Blot Analysis. Western analysis was performed as described previously with slight modifications. Briefly, cells were harvested and lysed in triple-detergent RIPA buffer containing 50 mM Tris-HCl (pH 8.0), 150 mM NaCl, 1% Nonidet P-40, 0.5% deoxycholate, 0.1% SDS, 1 mM EDTA, and Complete Proteinase Inhibitor Mixture Tablets (Roche Applied Science, Pleasanton, Calif.). Protein quantity was determined by Bradford Assay. Forty μg of protein was electrophoresed on a 12% SDS-PAGE gel and transferred to a nitrocellulose membrane. The membrane was blocked with 5% nonfat milk powder at room temperature for 1 hr and incubated with anti-β-tubulin (1 pg/ml, Sigma-Aldrich) at 4° C. overnight. After washing, the membrane was incubated with monoclonal anti-HuR clone 3A2 antibody (1 μg/ml) at room temperature for 1 hr. The secondary antibody used was sheep anti-mouse Ig horse radish peroxidase (diluted 1:4000) (GE Healthcare, Piscataway, N.J.) incubated at room temperature for 1 hr. For detection of VEGFa and TSP1 from cells, 100 μg of cell lysate was harvested, electrophoresed and transferred as above. For detection of VEGFa and TSP1 from tumors, protein was extracted by grinding snap frozen tumors with mortar and pestle and lysed in triple-detergent RIPA lysis buffer with protease inhibitors and 100 pg of protein was used for analysis. Membranes were probed with anti-TSP1 (Abcam) (7.5 pg/ml) or anti-VEGFa (Abcam) (1 pg/ml) and anti-β-tubulin (Sigma-Aldrich) (1 pg/ml). The secondary antibodies used were sheep anti-mouse Ig horse radish peroxidase (1:4000) (GE Healthcare) or donkey anti-rabbit Ig horse radish peroxidase (1:4000) (GE Healthcare) for VEGFa and TSP1, respectively. Specific proteins were detected using chemiluminescence (GE Healthcare). HuR, TSP1, and VEGFa levels were determined using Bio-Rad's Quantity One software (Bio-Rad) normalizing to β-tubulin. Anti-HuR 3A2 hybridoma was kindly provided by Joan Steitz (Yale School of Medicine).

In vitro growth and cell cycle assay. For in vitro cell proliferation, cells were trypsinized and counted using Trypan blue exclusion dye. Fifty thousand cells for both clones were seeded in a 24-well plate with 1 ml of media. Three wells from each clone were trypsinized and counted with a hemocytometer on three consecutive days using different wells each day. Four independent counting assays were performed to generate growth curves. For cell cycle analysis, cells were grown to 70% confluency, trypsinized, washed, fixed and permeabilized overnight in ethanol at 4° C. Cells were washed the following day and resuspended in PBS with 0.2 mg/mi RNase A (Sigma-Aldrich) and 10 mg/ml propidium iodide (Sigma-Aldrich). Cells were analyzed on FACScan (BD Biosciences, San Jose, Calif.) and cell cycle analysis was performed using Cell Quest software (BD Biosciences). Histogram is representative of three independent experiments.

Mice tumor inoculations and measurements. Athymic nude mice were purchased from Harlan and maintained in pathogen-free environments. For tumor inoculations, 100 μl of a 1:1 mixture of Matrigel (BD Biosciences) and RPMI 1640 (GIBCO®) containing 1×10⁶ MDA-MB-231 cells, expressing either pZeo HA HuR, pZeo empty vector or wild-type clones, were injected into the left or right mammary pad. Tumor volumes were calculated using calipers by measuring the length, width and depth of the tumor and using the formula: L×W×D×0.5. Experimental procedures performed on these animals were conducted according to the guidelines of the University of Missouri Columbia Animal Care and Use Committee.

Longitudinal MRI investigation and tumor volume analysis. Magnetic Resonance Imaging (MRI) was performed using a 7T/210 mm Varian Unity Inova MRI system equipped with a gradient insert (400 mT/m, 115 mm I.D.) and a quadrature driven birdcage coil (38 mm I.D) (Varian Inc., Palo Alto, Calif.). Mice were anesthetized with 1-2% isoflurane in oxygen via a nose cone over the entire imaging period. A respiratory sensor was placed on the abdomen for respiratory gating and monitoring of vital signs. Body temperature was maintained at 37° C. with warm air circulating in the magnet bore. Physiological monitoring was performed using a Physiological Monitoring System (SA Instruments, Inc., Stony Brook N.Y.). Three mice were imaged weekly for 5 weeks to monitor tumor growth. Mice were imaged to obtain axial planes using multi-slice spin echo T1-weighted (T1 W) imaging sequence applied with fat-saturation pulse to suppress the strong signals from fatty tissues in the chest. Spin-echo diffusion-weighted imaging (DWI) was performed at week 4 to assess the tumor tissue viability, i.e., necrotic tissue or solid tumor tissue. The following parameters were used: fat-saturated T1 W: repetition time (TR)/echo time (TE)=650 msec/18 msec, 21-25 slices, slice thickness=0.8 mm with no gap, image matrix size=512×256, field of view (FOV)=3 cm×4 cm, number of averages=6; DWI: TR/TE=2200 msec /37.2 msec, b-value=1063 s/mm2, number of averages=2. Tumor volume measurements were performed using fat-saturated TI W image stacks. The tumors were manually segmented using VnmrJ software (Varian Inc.) to obtain the tumor volume in cm³. DW images at week 4 were used to differentiate between necrotic tissues and solid tumor tissues.

Tumor harvest. Mice were sacrificed and tumors were removed, weighed, and either snap-frozen in liquid nitrogen, placed in buffered formalin (10% v/v), or digested and reestablished in tissue culture. Tumor digestions were performed by mincing tumors with a scalpel, digesting with collagenase (Sigma-Aldrich) and hyaluronidase (Sigma-Aldrich), and filtering through 0.70 micron filter. Cells extracted from tumors were grown in standard media as described above.

RNA purification and real-time PCR. RNA was extracted from tumors by grinding snap frozen tumors with mortar and pestle in Trizol reagent (Invitrogen™). The manufacturer's protocol was followed for the remainder of the extraction. For real-time PCR, 1 μg of RNA was reverse-transcribed and the resulting cDNA was divided into 15 reactions using four sets of primers, in triplicate, for real-time PCR using SuperScript III two-step qRT-PCR with SYBR green (Invitrogen™). Primers for specific gene targets are shown in Table 1. All real-time PCR reactions were performed using the Applied Biosystems StepOne real-time PCR system. Results were analyzed using the comparative CT method. GAPDH was used as an endogenous reference.

TABLE 1
List of Primer Pair Nucleotide Sequences

| Primer Name | Sequence | SEQ ID NO |
|---|---|---|
| VEGFa sense | 5′-TTT CTG CTG TCT TGG | (SEQ ID NO: 1) |
| | GTG CAT TGG-3′ | |
| VEGFa | 5′-ACC ACT TCG TGA TGA | (SEQ ID NO: 2) |
| antisense | TTC TGC CCT-3′ | |
| TSP1 sense | 5′-TTC CGC CGA TTC CAG | (SEQ ID NO: 3) |
| | ATG ATT CCT-3′ | |
| TSP1 | 5′-ACG AGT TCT TTA CCC | (SEQ ID NO: 4) |
| antisense | TGA TGG CGT-3′ | |
| HIF1α sense | 5′-TTG GCA GCA ACG ACA | (SEQ ID NO: 5) |
| | CAG AAA CTG-3′ | |
| HIF1α | 5′-TTG AGT GCA GGG TCA | (SEQ ID NO: 6) |
| antisense | GCA CTA CTT-3′ | |
| GAPDH sense | 5′-AGC CTC AAG ATC ATC | (SEQ ID NO: 7) |
| | AGC AAT GCC-3′ | |
| GAPDH | 5′-TGT GGT CAT GAG TCC | (SEQ ID NO: 8) |
| antisense | TTC CAC GAT-3′ | |

Microarray. For RNA amplification and labeling, 0.5 μg of total RNA was used to make the biotin-labeled antisense RNA (aRNA) target using the Illumina TotalPrep RNA amplification kit (Ambion, Austin, Tex.) according to the manufacturer's protocol. Briefly, total RNA was reverse transcribed to first strand cDNA with a oligo(dT) primer bearing a 5′-T7 promoter using ArrayScript reverse transcriptase. The first strand cDNA underwent second-strand synthesis and clean-up to become the template for in vitro transcription. The biotin-labeled aRNA was synthesized using T7 RNA polymerase with biotin-NTP mix and purified. One and one-half μg of aRNA was hybridized to the human Illumina BeadChip (47,000 genes) array at 58° C. for 20 hrs. After hybridization, the chips were washed and stained with streptavidin-C3. The image data was acquired by BeadArray reader (Illumina, San Diego, Calif.).

Histology and Immunohistochemistry. Tissue was routinely processed, formalin-fixed and embedded in paraffin blocks for hematoxylin-and-eosin-staining and immunohistochemistry. Immunostaining was performed using the avidin-biotin-peroxidase complex method as previously described (refs below). Briefly, deparaffinized, rehydrated 5 μm sections were rinsed in wash buffer (DAKO, Carpinteria, Calif.) and heated for 20 min in either 10 mmol/L citrate buffer (pH 6.0) for all antibodies used, or in Tris/EDTA (pH 9.0) for TSP-1 immunolabeling. Slides were cooled for 20 min, treated with 3% hydrogen peroxide to inactivate endogenous peroxidase activity, and rinsed for 20 min with 5% bovine serum albumin. After rinsing, slides were incubated for 60 min at room temperature with one of the following antibodies: anti-cleaved caspase-3 antibody (1:100 dilution, rabbit anti-human cleaved caspase-3 polyclonal antibody [2305-PC-100], Trevigen, Gaithersburg, MD); anti-CD34 (1:50 dilution, rat anti-mouse CD34 monoclonal antibody [68158, MEC 14.7], Abcam, Cambridge, Mass.); antiVEGF (1:200 dilution, rabbit anti-human VEGF-A polyclonal antibody [sc-152] Santa Cruz Biotechnology, Inc., Santa Cruz, Calif.); and anti-TSP-1 (1:400 dilution, mouse monoclonal antibody [clone A6.1, MS-420-P1, Thermo Fisher Scientific, Fremont, Calif.). Slides labeled with anti-CD34 or TSP-1 were incubated for 30 minutes with a biotinylated secondary antibody (swine anti-rat IgG [DAKO] for CD-34 and rabbit anti-mouse IgG [DAKOJ for TSP-1) followed by streptavidin-linked horseradish peroxidase product (DAKO) for 30 min. Cleaved caspase-3 and VEGF slides were incubated with horseradish peroxidase-labeled polymer conjugated to anti-rabbit immunoglobulin (EnVision™, DAKO). PBS was used for rinsing between steps. Bound antibodies were visualized following incubation for 3-5 min with one of two peroxidase substrates: DAB (3,3′-diaminobenzidine solution [0.05% with 0.015% H₂O₂ in PBS; DAKO]) or NovaREDT™ (Vector Labs, Burlingame, Calif.).

RNA purification and real-time PCR for metastasis study. RNA was extracted from cell lines grown in tissue culture by adding 1 ml of Trizol reagent (Invitrogen) to adherent cells and following manufacturer's protocol. For tumors, RNA was extracted from tumors by grinding snap frozen tumors with a mortar and pestle in Trizol reagent (Invitrogen). The manufacturer's protocol was followed for the remainder of the extraction. For real-time PCR, 1 pg of RNA was reverse transcribed and the resulting cDNA was divided into 12 reactions, comprising three sets of primers done in triplicate for real-time PCR using SuperScript Ill two-step qRT-PCR with SYBR green (Invitrogen). Primers were specific for CXCR4, SOX4, and GAPDH. All real-time PCR reactions were performed using Applied Biosystems StepOne real-time PCR system. Results were analyzed using the comparative CT method. GAPDH was used as an endogenous reference control.

Senescence assay. β-galactosidase staining was performed on tissue sections from frozen tumors to assay for senescence in breast cancer tumors, using the senescence cells histochemical staining kit (Sigma-Aldrich) according to the manufacturer's instructions.

Statistical Analysis of Microarray Data. Analysis of microarray gene expression data was primarily performed using the Linear Models for Microarray Data (limma) package and the lumi package, available through the Bioconductor project [Gentleman et al., Genome Biol 2004, 5:80] for use with R statistical software. Data quality was examined by looking at quality control metrics produced by Illumina's software (BeadStudio v3.1.3.0, Gene Expression Module 3.3.$). The data were then exported for further analyses with R statistical software. Image plots of each array were examined for spatial artifacts, and there was no evidence of systematic effects would be indicative of technical problems with the arrays. Within limma, quantile normalization was used for between chip normalization. Quality control statistics were computed using a variety of Illumina's internal control probes that are replicated on each array. Probes which were considered “not detectable” across all samples were excluded from further statistical analyses to reduce false positive signals. The determination of “not detectable” was based upon the BeadStudio computed detection p-value being greater than 1%.

After pre-processing was completed, the statistical analysis was performed using moderated t-statistics applied to the log-transformed (base 2) normalized intensity for each gene. Because two measurements were taken from each mouse, the dependency between paired measurements was accounted for by a modified mixed linear model that treated each animal as a block. The within-block correlations were constrained to be equal between genes (Smyth, G K, Statistical Applications in Genetics and Molecular Biology: 2004, 3:1, Article 3), and then information was borrowed across genes to moderate the standard deviations between genes via an empirical Bayes method (Smyth, G K, et al., Bioinformatics 2005, 21:2067-2075). The contrast of interest computed and tested was the difference between overexpressor and control vector, which is equivalent to the fold change (overexpressor/control) because the data is on the log scale. For this contrast, we computed the aforementioned moderated t-statistics and corresponding nominal and adjusted p values, along with estimated log-odds ratios of differential expression.

Adjustments for multiple testing were made using the false discovery rate (FDR) method of Benjamini and Hochberg (Benjamini, Y, and Hochberg, Y, J. Royal Statistical Society 1995; 57:289-300). We chose 10% as our FDR-cutoff for declaring statistical significance, and a threshold at least 3-fold (up or down) for declaring a biologically significant change in expression. To facilitate interpretation in this report, log fold changes were transformed back to fold change on the data scale and log-odds ratios of differential expression were converted into probabilities of differential expression.

Gene ontology (GO) analyses were carried out on the list of genes that met the described criteria for statistical and biological significance. The purpose of the analyses was to test the association between Gene Ontology Consortium categories and the list of differentially expressed genes. In defining the gene universe for the analysis, non-specific filtering was used to increase statistical power without biasing the results. We started with all probes on the Illumina array which had both an Entrez gene identifier and a GO annotation, as provided in the lumiHumanAll.db annotation data package and GO.db annotation maps (built using data obtained from NCBI on 4/2108). This set was then reduced by excluding probes that exhibited little variability (interquartile range (IQR) of <0.1 on log2 scale) across all samples because such probes are generally not informative. Finally, for probes that mapped to the same Entrez identifier, a single probe was chosen in order to insure a subjective map from probe IDs to GO categories (via Entrez identifiers). This was necessary to avoid redundantly counting GO categories which produces false positives. Probes with the largest IQR were chosen to be associated with an Entrez identifier. Using this gene universe, GOstats was used to carry out conditional hypergeometric tests. These tests exploit the hierarchical nature of the relationships among the GO terms for conditioning. We carried out GO analyses for over-representation of biological process (BP), molecular function (MF), and cellular component (CC) ontologies, and computed the nominal hypergeometric probability for each GO category. These results were used to assess whether the number of selected genes associated with a given term was larger than expected, and a p-value cutoff of 0.01 was used. GO categories containing less than 10 genes from our gene universe were not considered to be reliable indicators, and are not reported.Statistics: All error bars represent standard error of the mean (±SEM). Probability values (p-value) were calculated using the two-tailed Student t test.

### Results

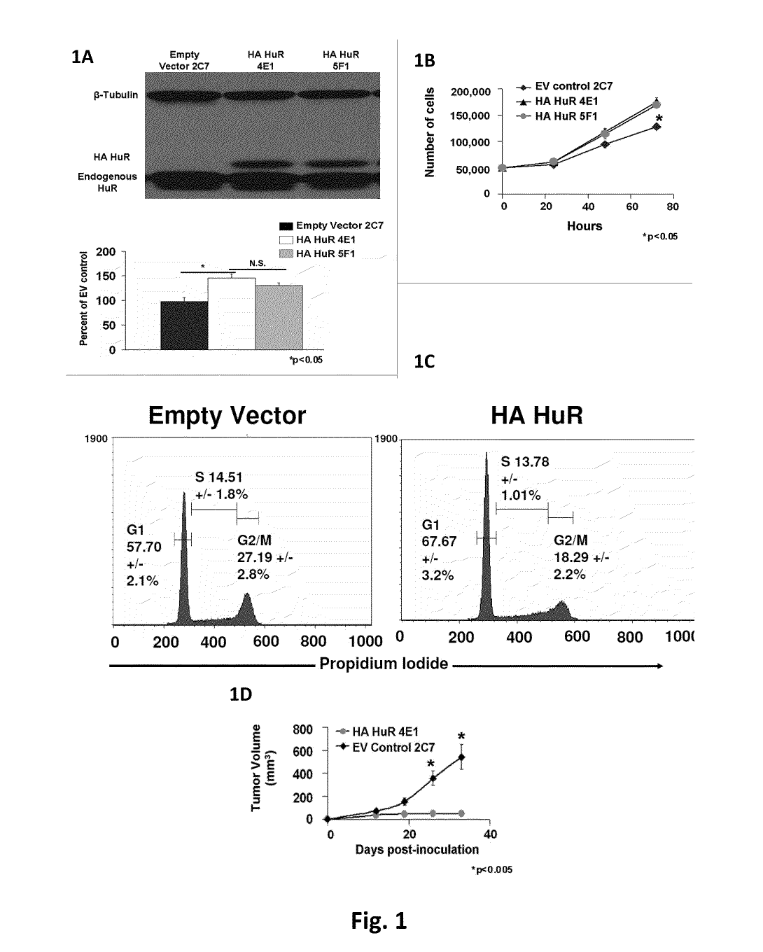

Over expression of HuR in MDA-MB-231 cells increases growth rates and alters cell cycle kinetics: To study the role of HuR expression in MDA-MB-231 ER− breast cancer we made individual clones which over expressed either epitope-tagged (HA) HuR or empty vector (EV) control and measured growth rates and cell cycle kinetics (FIG. 1). Overexpression of HA HuR resulted in accelerated cellular growth as determined by counting (FIG. 1B). When the cells were stained with propidiurn iodide, we noted an alteration in cell cycle kinetics. HA-HuR overexpressing cells had increased amounts of cells in G₁ (67 vs. 57%), as compared with empty vector controls. Conversely, HuR over expression also resulted in a compensatory decrease in G₂/M percentages (18% vs. 27%). We concluded, as expected, that HuR over expression resulted in increases in growth rates of MDA-MB-231 cells. We then investigated the effects of HuR over expression in vivo, using orthotopic xenograft animal models.

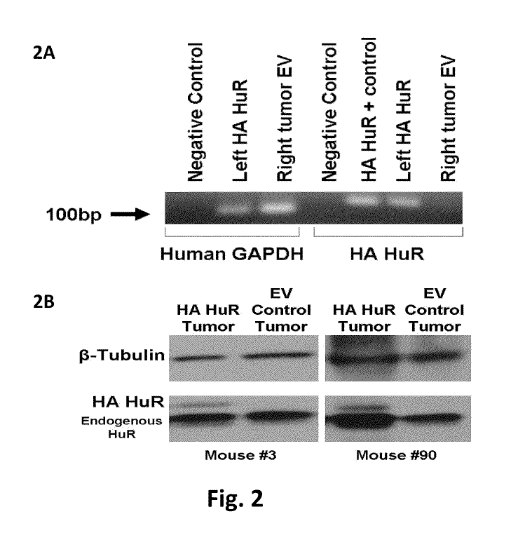

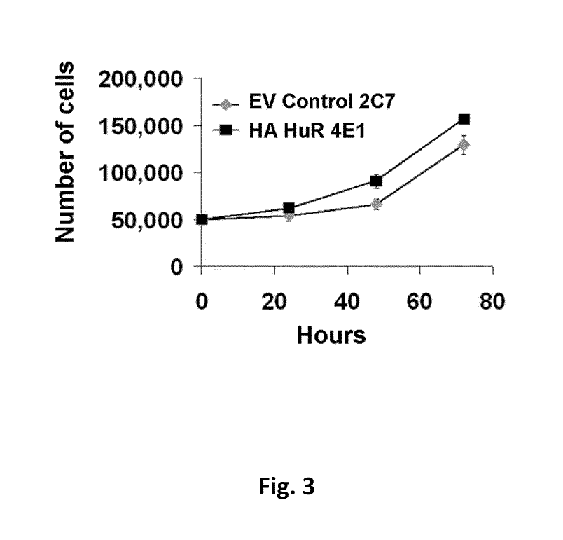

HuR over expression results in significantly reduced tumor growth and mass: The clones used in FIG. 1, empty vector (2C7) and over expresser HA-HuR (4E 1), were injected into the contralateral mammary fat pads of athymic nude mice. Tumor growth was assessed weekly by caliper measurements and followed in vivo by MRI scan. Surprisingly, tumors which over expressed HuR did not significantly grow, whereas EV control tumors increased significantly in tumor volume (FIG. 1D). The tumors were removed from the animals on day 35. Histological staining and RT-PCR were performed to determine whether any cells remained. Both control (2C7) and HuR (4E1) tumors had intact human GAPDH mRNA and tumor 4E I expressed the HA-HuR transgene (FIG. 2). We reestablished cell lines from tumors removed from animals to study their growth rates. We found that these reestablished cells had similar growth rates to parental cells prior to transplantation (FIG. 3).

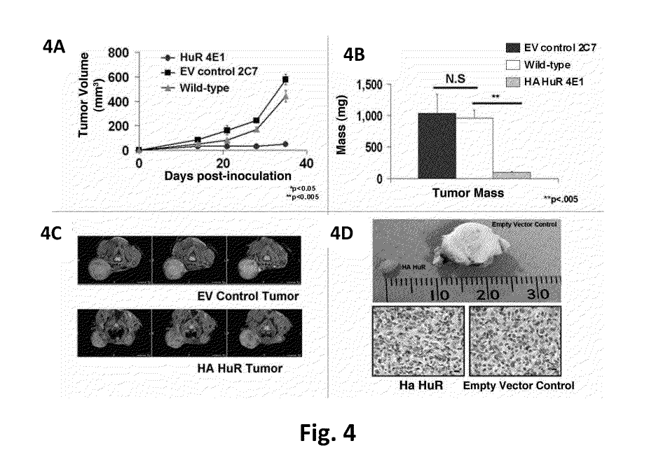

The experiments were repeated with wild-type, parental MDA-MB-231 cells to validate these findings. As seen in FIG. 4, both parental and control cells (2C7) grew at similar rates, whereas tumors which overexpressed HuR (4E I) did not appear to grow (FIG. 4A). Tumor mass was assessed, which showed that HuR overexpression resulted in a 90% reduction in growth (FIGS. 4B,4C). These results were confirmed by MRI scans, gross photographs, and microscopy (FIG. 4D). HA-HuR tumors appeared to be a gelatin capsule and the control tumors were a solid round mass. Cross sections of both revealed that the HA HuR tumors had a smooth, homogeneous, and glistening surface, while control tumors had a heterogeneous, yellow-white surface with a necrotic center (FIG. 4D). Both tumors contained viable cancer cells and similar morphology determined to be moderately to poorly differentiated carcinoma, consistent with the implanted MDA-MB-231 cells (FIG. 4D, lower panel). We concluded that HuR overexpression resulted in significantly smaller ER− tumors in animals.

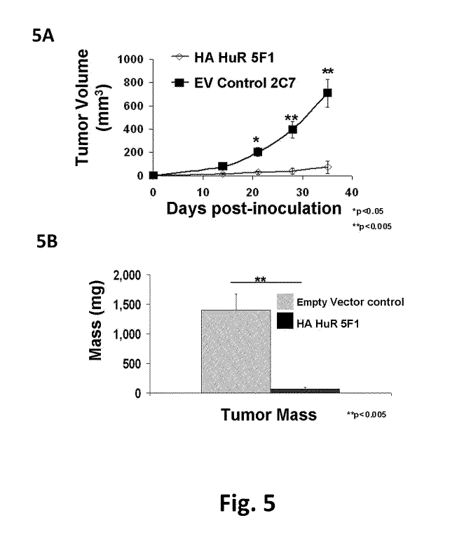

To verify that these results were not clonal, we repeated the orthotopic tumor injection experiments using a second HuR over-expressing clone (5F1), which in vitro grows similarly to the original over-expressing clone, 4E1 (FIG. 1B). As seen in FIG. 5, tumor 5F1 also exhibited retarded growth rates compared to empty vector controls and had a 90% reduction in mass (FIG. 3B). Taken together, these findings demonstrate that HuR overexpression in MDA-MB-231 cells resulted in significant reductions in tumor growth in a clonal-independent fashion.

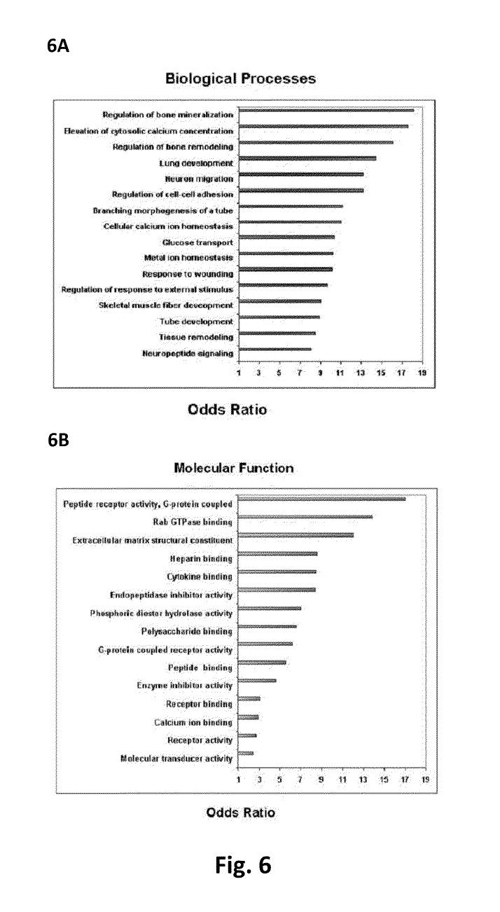

Gene ontology (GO) analysis of genes which are over expressed in HuR over expressing cells: In order to better understand the genes which may be involved in altering tumor growth in HuR over expressing MB-231 cells, we performed genome wide microarray analysis. As seen in Table 2 and FIG. 6{ }, many genes were over represented by odds ratio dealing with both biological processes as well as molecular function. Given the large numbers of genes, we decided to investigate the following three potential mechanisms to explain the large discrepancy seen in tumor growth: (1) increased apoptosis, (2) increases in senescence, and (3) alteration in angiogenesis.

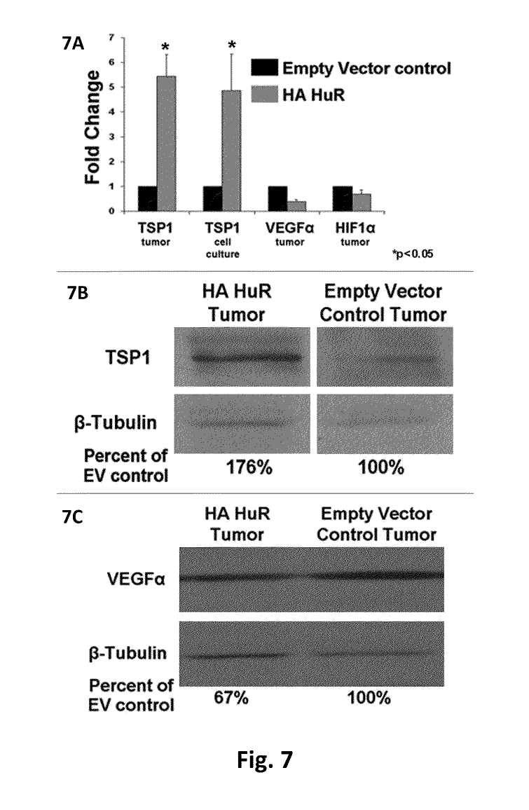

Tumors which over express HuR have decreased angiogenesis: We conducted experiments which targeted well known HuR target genes involved in angiogenesis which had previously been described in the literature: thrombospondin 1 (TSP1), VEGF, and HIF1α. We had also detected TSP1 as a gene of interest from the GO analysis (Table 2). We performed real time PCR to measure mRNA levels of TSP1, VEGF, HIF1α. As shown in FIG. 7A, HuR overexpression caused an increase in TSP1 mRNA and protein (FIG. 7B). Surprisingly, decreases in steady-state VEGF mRNA and protein levels were observed. Steady-state HIF1α mRNA levels did not appear to change (FIGS. 7A, 7B, and 7C).

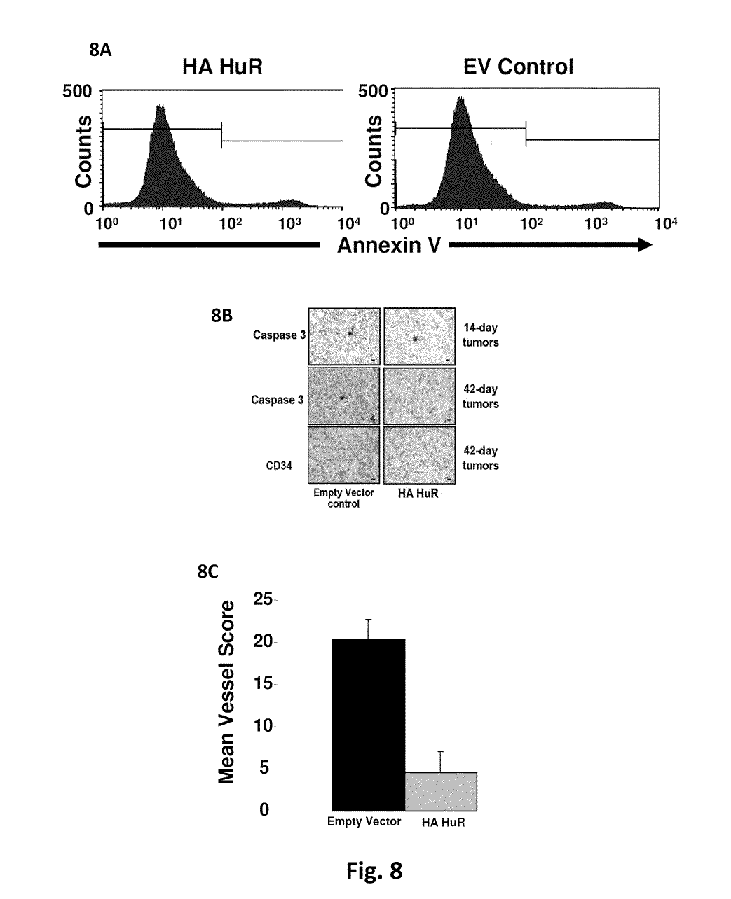

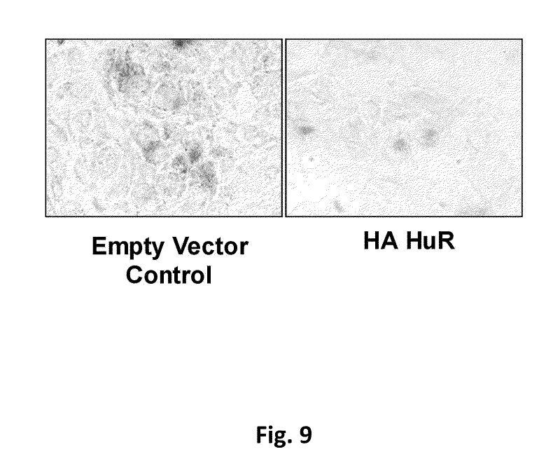

We next examined whether alterations in apoptosis could account for differences in tumor growth. As seen in FIG. 8A, there were no increases in apoptosis in HuR over expression tumors (right), as compared with EV controls. Increased apoptosis in EV tumors was mostly found in the necrotic centers of these samples. We measured the amount of blood vessels, however, and found a significant decrease in blood vessel formation in HA-HuR tumors, compared to EV controls (FIGS. 8B and 8C). No tortuous vessels were observed. These non-functional vessels are sometimes seen when there are perturbations in the DLL4-Notch signaling pathways. Measurement of senescence using β-galactosidase staining did not reveal any significant differences (see FIG. 9). Taken together, we conclude that overexpression of HuR in MB-231 tumors interferes with angiogenesis by overexpression of an anti-angiogenic factor, TSP1, and by down-regulation of VEGF, and perhaps, HIF1α.

### Discussion

MDA-MB-231 ER− cells, which overexpress HuR, have increased growth rates and alterations in their cell cycle kinetics. Specifically, MB-231 cells which overexpress HuR, have increases in the G₁ phase of the cell cycle, which is consistent with earlier observations (Lopez de Silanes I, et al. Oncogene 2003; 22:7146-54). A plausible explanation is HuR-induced stabilization of cyclin B1, the pivotal cyclin involved in transition of cells from G₂ to the M phase of the cell cycle.

Surprisingly, when these same cells were transplanted into athymic nude mice, we observed that overexpression of HuR resulted in a 90% reduction in tumor size. These results were confirmed by measuring volume and mass of the tumors. These results were also confirmed by serial MRI scans during the experimental period and evaluating data from two independent clones. Cells harboring empty vector (EV) controls and parental wild-type MDA-MB-231 cells had similar growth rates, generating tumors that were much larger than those formed by cells that overexpressed HA-HuR.

We searched for mechanisms to explain these surprising findings, since HuR overexpression in other systems results in larger, more robust tumor growth. Cross sections of EV and HA-HuR tumors that were stained had similar morphologies, which were characteristic of a poorly undifferentiated carcinomas found in ER− breast cancers. The HuR transgene was also expressed in smaller tumors. There was no evidence of inflammatory infiltrates seen in either tumor, however. As expected, increased apoptosis was observed in the centers of EV tumors, since these regions are relatively more hypoxic. These results ruled out apoptosis as mechanism of reduced tumor growth in the HA HuR tumors. We also ruled out senescence as mechanism, where there is less β-galactosidase staining in the smaller tumors (FIG. 9).

Earlier observations on the role of HuR in controlling angiogenesis through interactions with VEGF and HIF1α mRNAs, led us to investigate the relationship between HuR overexpression and pro-angiogenic factors (Levy N S, et al., J Biol Chem 1998; 273:6417-23; Galban S, et al., Mol Cell Biol 2008; 28:93-107). To our surprise, there was a statistically significant decrease in VEGF mRNA and protein expression, but no increase in HIF1α mRNA expression. As expected, increased HuR expression was correlated with increased TSP 1 expression at both the mRNA and protein levels. TSP1 is well known antiangiogenic factor and it has been described to be regulated by HuR. Quantitation of neo-angiogenesis by staining confirmed our hypothesis that HuR overexpression significantly decreases new blood vessel formation. These observations may explain, in part, why these tumors are much smaller than EV controls.

HuR has been described to stabilize TSP1, and VEGF mRNAs resulting in greater levels and increased protein expression. Its relationship with HIF1α, however, is more complex. HuR binds to AU-rich (ARE) regions in the 5′-UTR of HIF1α, instead of its 3′-UTR, even though both regions of the molecule possess AREs, causing translational upregulation in HIF1α protein synthesis without altering mRNA levels. We do not know whether HuR overexpression in our system is affecting HIF1α protein production, although it is known to be the major transcriptional factor involved in VEGF mRNA synthesis. These results indicate that HuR overexpression in ER− breast cancer provides a “double blow” to these cells, affecting angiogenesis by increasing TSP1, an inhibitor, and decreasing VEGF, a facilitator of angiogenesis. The effect of HuR upon HIF1α is less clear. The net result, however, is a substantial decrease in tumor size. Our experiments are reproducible, when each mouse is compared with its cohorts within individual and duplicate experiments, and clone-independent, as we obtain similar results when using different overexpression clones.

HuR induced anti-angiogenic effects are not completely understood at a molecular level, but are believed to involve interactions between HuR and microRNAs. HuR has been shown to recruit let-7 miRNA to c-myc mRNA to translationally suppress its expression (Kim H H, et al., Genes Dev 2009; 23:1743-8). While we do not have any direct evidence that the HA tag located at the amino terminal end of HuR affects its distribution or targeting of to its mRNA targets, Katsanou V, et al., (Mol Cell 2005; 19:777-89) have observed that when the same epitope tag was used to make a transgenic mouse which overexpresses HA-HuR in macrophages, they did not see alterations in the nuclear or cytoplasmic distribution of HuR or alterations in the binding of HuR to its mRNA targets. Experiments performed under hypoxic conditions, where tumors form in animals, compared to experiments performed in in vitro studies under normal oxygen conditions, may account for some of the observed differences.

Table 2 lists the tumor microarray results revealing 48 annotated genes upregulated in the HA HuR overexpressing tumors as compared to EV control tumors. The 48 genes are up-regulated 3-fold or greater in the HA-HuR tumors compared to EV control tumors (false discovery rate <1%), and also have a probability of differential expression >80% based on a Bayesian analysis.

TABLE 2
Genes up-regulated in tumors that overexpress HuR

| Fold change | Entrez ID | Gene Name |
|---|---|---|
| 21.52 | 79191 | IRX3/iroquois homeobox 3 |
| 16.87 | 4256 | MGP/matrix Gla protein |
| 12.21 | 1278 | COL1A2/collagen, type I, alpha2 |
| 9.35 | 8714 | ABCC3/ATP -binding cassette, sub-family C (CFTR/MRP), member 3 |
| 7.58 | 221476 | PI16/peptidase inhibitor 16 |
| 7.46 | 972 | CD74/CD74 molecule, major histocompatibility complex, class II |
| | | invariant chain |
| 6.90 | 2202 | EFEMP1/EGF-containing fibulin-like extracellular matrix protein 1 |
| 6.84 | 5265 | SERPINA1/serpin peptidase inhibitor, clade A (alpha-1 antiproteinase, |
| | | antitrypsin), member 1 |
| 6.77 | 3399 | ID3/inhibitor of DNA binding 3, dominant negative helix-loop-helix |
| | | protein |
| 6.63 | 6275 | S100A4/S100 calcium binding protein A4 |
| 5.85 | 57537 | SORCS2/sortilin-related VPS10 domain containing receptor 2 |
| 5.07 | 6275 | S100A4 |
| 5.07 | 1831 | TSC22D3/TSC22 domain family, member 3 |
| 4.87 | 51313 | C4orf18/chromosome 4 open reading frame 18 |
| 4.87 | 4061 | LY6E/lymphocyte antigen 6 complex, locus E |
| 4.77 | 3108 | HLA-DMA/major histocompatibility complex, class II, DM alpha |
| 4.61 | 10507 | SEMA4D/sema domain, immunoglobulin domain (Ig), transmembrane |
| | | domain (TM) and short cytoplasmic domain, (semaphorin) 4D |
| 4.46 | 7052 | TGM2/transglutaminase 2 (C polypeptide, protein-glutamine-gamma- |
| | | glutamyltransferase) |
| 4.46 | 1728 | NQO1/NAD(P)H dehydrogenase, quinone 1 |
| 4.43 | 9022 | CLIC3/chloride intracellular channel 3 |
| 4.42 | 7057 | THBS1/thrombospondin 1 |
| 4.36 | 7857 | SCG2/secretogranin II (chromogranin C) |
| 4.29 | 4599 | MX1/myxovirus (influenza virus) resistance 1, interferon-inducible |
| | | protein p78 (mouse) |
| 4.24 | 22998 | LIMCH1/LIM and calponin homology domains 1 |
| 4.14 | 3371 | TNC/tenascin C |
| 4.13 | 6376 | CX3CL1/chemokine (C-X3-C motif) ligand 1 |
| 4.1 | 199 | AIF1/allograft inflammatory factor 1 |
| 4.05 | 652 | BMP4/bone morphogenetic protein 4 |
| 3.94 | 7058 | THBS2/thrombospondin 2 |
| 3.86 | 26227 | PHGDH/phosphoglycerate dehydrogenase |
| 3.78 | 144165 | PRICKLE1/prickle homolog 1 (Drosophila) |
| 3.76 | 4147 | MATN2/matrilin 2 |
| 3.75 | 8140 | SLC7A5/solute carrier family 7 (cationic amino acid transporter, y+ |
| | | system), member 5 |
| 3.54 | 8460 | TP ST1/tyrosylprotein sulfotransferase 1 |
| 3.48 | 64764 | CREB3L2/cAMP responsive element binding protein 3-like 2 |
| 3.44 | 3397 | ID1/inhibitor of DNA binding 1, dominant negative helix-loop-helix |
| | | protein |
| 3.42 | 9516 | LITAF/lipopolysaccharide-induced TNF factor |
| 3.4 | 55971 | BAIAP2L1/BAI1-associated protein 2-like 1 |
| 3.36 | 80063 | ATF7IP2/activating transcription factor 7 interacting protein 2 |
| 3.26 | 1021 | CDK6/cyclin-dependent kinase 6 |
| 3.25 | 7100 | TLR5/toll-like receptor 5 |
| 3.17 | 94121 | SYTL4/synaptotagmin-like 4 |
| 3.16 | 8622 | PDE8B/phosphodiesterase 8B |
| 3.15 | 55616 | DDEFL1/ArfGAP with SH3 domain, ankyrin repeat and PH domain 3 |
| 3.13 | 30818 | KCNIP3/Kv channel interacting protein 3, calsenilin |
| 3.13 | 3430 | IFI35/interferon-induced protein 35 |
| 3.07 | 29887 | SNX10/sorting nexin 10 |
| 3.01 | 23052 | ENDOD1/endonuclease domain containing 1 |

These observations suggest that overexpression of HuR in ER− breast cancer cells increases TSP1 expression, but decreases VEGF expression, and that one or both of these events are linked to related to metabolic changes that lead to a substantial decrease in tumor size. Thrombospondin 2 (TSP2), a potent anti-angiogenic factor, is also significantly up-regulated (see Table 2). These results are clone-independent and highly reproducible, when each mouse is compared to its cohorts within individual and duplicate experiments.

In view of increasing evidence that cancer cells readily overcome therapies designed to target the expression or activity of a single gene product, approaches that modulate the expression of RBPs, such as HuR, may facilitate the treatment of ER− tumors, by simultaneously interfering with a variety of key metabolic steps involved in neo-angiogenesis.

### Example 2

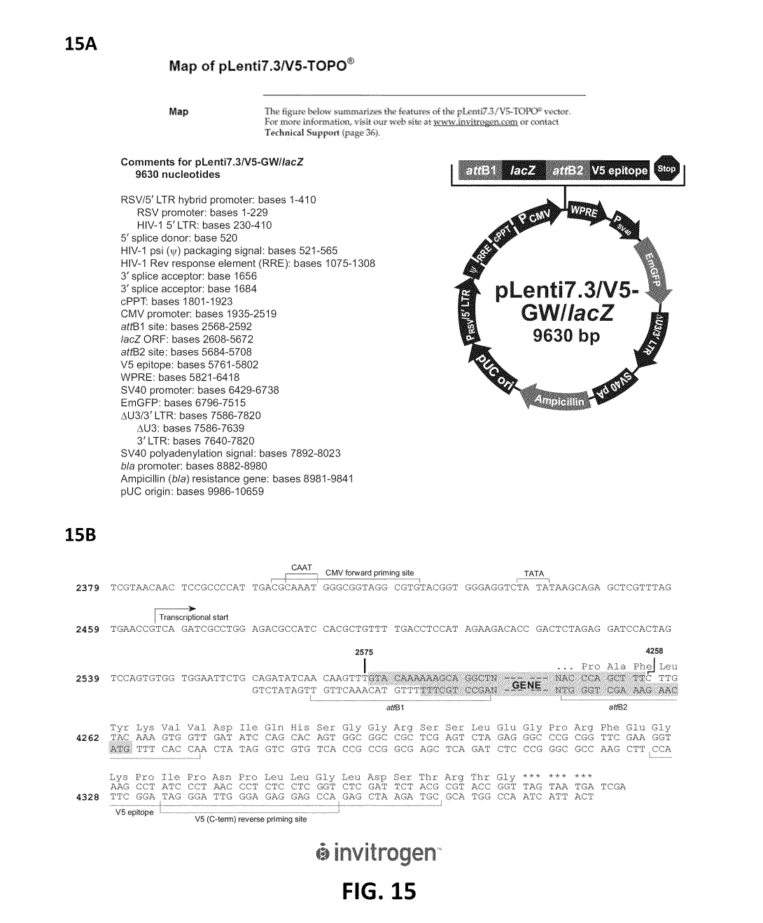

A lentivirus vector comprising a gene cassette containing an HuR gene (SEQ ID NO: 9, encoding HuR, SEQ ID NO: 10) operably-linked to a promoter was constructed and used to test whether over-expression of HuR inhibits tumor growth in MDA-MB-231 cells. A virus comprising a sequence encoding a hemagglutinin(HA)-tagged human HuR was constructed by amplifying the human HuR gene using forward primer encoding the HA tag (underlined in SEQ ID NO 11) and a reverse primer (SEQ ID NO: 12) positioned at the 3′ end of the human HuR gene, which was cloned into the plasmid pLenti7.3 using a TOPO® cloning kit provided by Invitrogen as shown in FIG. 15.

Forward TOPO primer:
(SEQ ID NO: 11)
5′-CACC ATG TAC CCA TAC GAT GTT CCA GAT TAC GCT
CTT ATGTCTAATGGTTATGAAGAC-3′
Reverse TOPO Primer:
(SEQ ID NO: 12)
5′-TTATTTGTGGGACTTGTTGGT-3′
HA sequence:
(SEQ ID NO: 13)
5′-atg tac cca tac gat gtt cca gat tac gct ctt-3′

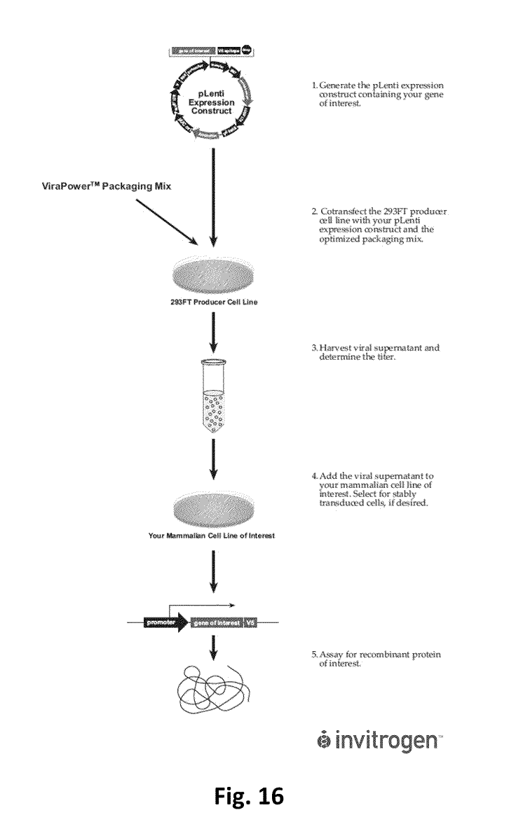

Lentiviral particles were prepared by packaging recombinant lentiviral DNAs in 293FT cells using a ViraPower Lentiviral Expression Systems kit (Invitrogen) following instructions provided by the manufacturer (FIG. 16). MB-231 cells were seeded at a density of 100,000 cells in 100 mm tissue culture plates with 10 ml of media. One day later, recombinant lentiviral stocks capable of expressing green fluorescent protein (GFP) and β-galactosidase, or GFP and HA-tagged HuR, were added to the cells at a multiplicity of infection (MOI) of 10, along with polybrene (8 pg/ml) (Sigma-Aldrich Corp, St. Louis, Mo.) to facilitate uptake of the viral particles. After five days, the cells were harvested after trypsinization, and sorted using GFP expression as a cell marker, using a BD FACSDiva cell sorting device (BD Bioscience). The cells were cloned by limiting dilution, and GFP expression was assessed with a FACScan device (BD Bioscience) using Cell Quest software (BD Bioscience). GFP expression was >98% in a homogenous cell population.

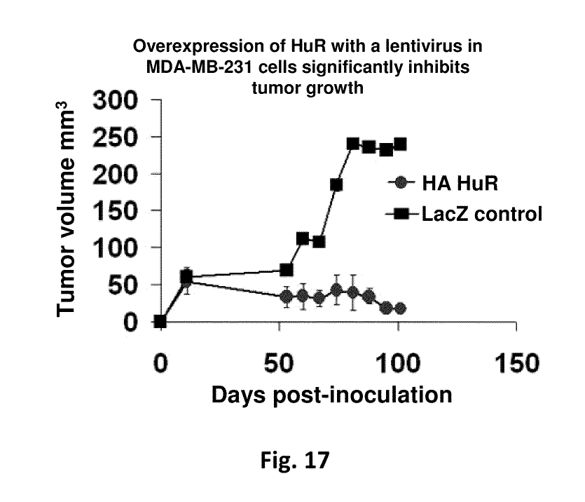

MDA-MB-231 cells infected with a lentivirus that over-expressed HA HuR showed significantly reduced tumor volume (mm³) and growth starting at five weeks post-inoculation and continuing for fourteen weeks when compared to MDA-MB-231 infected with a lentivirus expressing LacZ control (FIG. 17). Five animals per group were used. p<0.05.

### Example 3

A lentivirus vector containing a gene cassette that comprising a nucleotide sequence encoding a small hairpin RNA (shRNA) targeting HuR was also constructed and used to test whether under-expression of HuR with a lentivirus expressing a shRNA targeting HuR in MDA-MB-231 cells would inhibits tumor growth. The software program PSICOOLIGOMAKER v1.5 (web.mit.edu/ccr/labs/jacks) was used to identify optimal shRNA sequences that would target HuR. Multiple sequences were tested, and a sequence designated shRNA H760 (SEQ ID NO: 14), was chosen for detailed analysis.

| (SEQ ID NO 14) | |
|---|---|
| sh RNA H760 | 5′-GGATCCTCTGGCAGATGT-3′ |

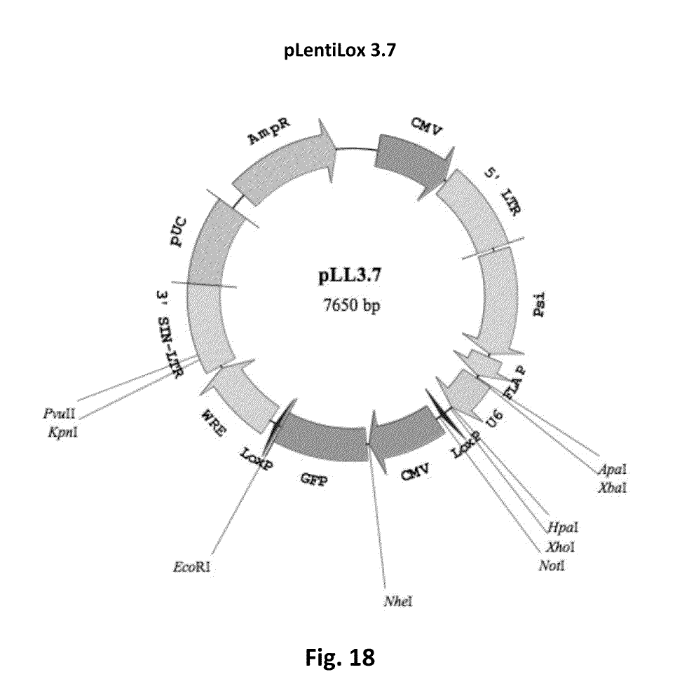

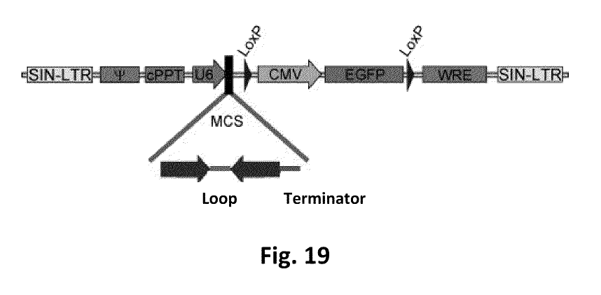

Sense and anti-sense DNAs comprising stem loops to create the shRNA hairpin, were synthesized (Integrated DNA-Technologies, Inc, IDT, Coralville, Iowa), annealed, and then cloned into the HpaI and XhoI restriction sites in the Lentilox pIl3.7 vector (ATCC) (FIG. 18). The sequence of the recombinant vector was verified, and lentiviral particles were prepared by packaging the viral DNAs in 293FT cells using a ViraPower Lentiviral Expression Systems kit (Invitrogen) according to instructions provided by the manufacturer (FIG. 19). MB-231 cells were seeded at a density of 100,000 cells in 100 mm tissue culture plates with 10 ml of media. One day later, recombinant lentiviral stocks capable of expressing GFP and no shRNA (empty lentilox control), or GFP and HuR shRNA H760, were added to the cells at a multiplicity of infection (MOI) of 10, along with polybrene (8 μg/ml) (Sigma-Aldrich Corp, St. Louis, Mo.) to facilitate uptake of the viral particles. After five days, the cells were harvested after trypsinization, and sorted using GFP expression as a marker using a BD FACSDiva cell sorting device (BD Bioscience). The cells were cloned by limiting dilution, and GFP expression was assessed with a FACScan device (BD Bioscience) using Cell Quest software (BD Bioscience). GFP expression was >98%, indicative of a homogenous cell population.

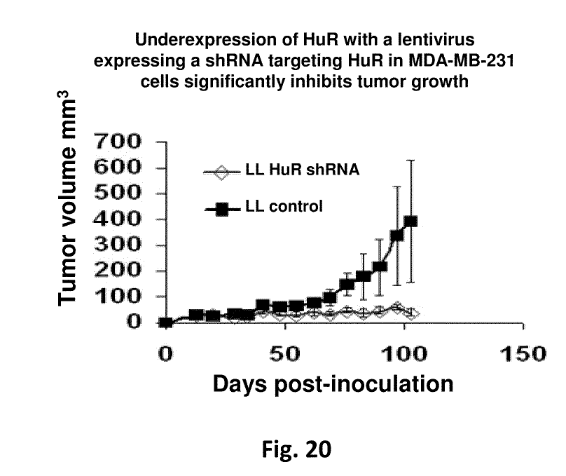

MDA-MB-231 cells infected with a lentivirus expressing an shRNA knocking down HuR (LL HuR shRNA) showed significantly reduced tumor volume (mm³) and growth starting at seven weeks post-inoculation and continuing for fourteen weeks when compared to MDA-MB-231 infected with a lentivirus expressing no shRNA (LL control) (FIG. 20). Five animals per group were used. p<0.05.

While the preferred embodiments of the invention have been illustrated and described in detail, it will be appreciated by those skilled in the art that that various changes can be made therein without departing from the spirit and scope of the invention. Accordingly, the particular arrangements disclosed are meant to be illustrative only and not limiting as to the scope of the invention, which is to be given the full breadth of the appended claims and any equivalent thereof. All references, patents, or applications cited herein are incorporated by reference in their entirety, as if written herein.

What is claimed is:

1. A method of inhibiting the replication or metastasis of cancer cells comprising contacting cells with a therapeutically-effective amount of an HuR-modulating agent.

2. The method of claim 1, wherein the HuR-modulating agent increases or decreases the level of expression of the RNA-binding protein HuR by than three-fold in a sample of cancer cells contacted with the HuR-modulating agent compared to control sample of cancer cells not contacted with the HuR-modulating agent.

3. The method of claim 2, wherein the level of expression of HuR is increased.

4. The method of claim 2, wherein the level of expression of HuR is decreased.

5. The method of claim 1, wherein the HuR modulating agent treats an HuR-mediated disease.

6. The method of claim 5, wherein said HuR-mediated disease is cancer.

7. The method of claim 6, wherein the cancer is breast cancer.

8. The method of claim 7, wherein the cancer cells are estrogen receptor negative breast cancer cells.

9. The method of claim 1, wherein the HuR-modulating agent comprises a single- or double-stranded nucleic acid comprising an HuR gene, or fragment thereof, operably-linked to a promoter active in cancer cells.

10. The method of claim 9, wherein said nucleic acid is single-stranded.

11. The method of claim 10, wherein said nucleic acid is single-stranded RNA.

12. The method of claim 11, wherein said single-stranded RNA is packaged in a virus.

13. The method of claim 11, wherein said virus is a retrovirus.

14. The method of claim 12, wherein said retrovirus is a lentivirus.

15. The method of claim 9, wherein said nucleic acid is double-stranded.

16. The method of claim 15, wherein said nucleic acid is double-stranded DNA.

17. The method of claim 16, wherein said double-stranded DNA is linear.

18. The method of claim 17, wherein said linear double-stranded DNA is packaged in a virus.

19. The method of claim 15, wherein said double-stranded DNA is circular.

20. The method of claim 19, wherein said circular double-stranded DNA is a plasmid.

21. The method of claim 19, wherein said circular double-stranded DNA is a packaged in a virus.

22. The method of claim 9, wherein said HuR gene, or fragment thereof, encodes an HuR polypeptide, or a fragment or variant thereof, capable of binding to mRNAs encoded by one or more genes involved in angiogenesis or metastasis.

23. The method of claim 22, wherein the level of expression of the HuR polypeptide, or a fragment of variant thereof, is increased in the cancer cells.

24. The method of claim 9, wherein said HuR gene, or fragment thereof, is operably-linked to the promoter active in cancer cells in an anti-sense direction.

25. The method of claim 24, wherein the level of expression of HuR is decreased in the cancer cells.

26. A composition for inhibiting the replication or metastasis of cancer cells comprising a therapeutically-effective amount of an HuR modulating agent.

27. The composition of claim 26, wherein the HuR-modulating agent increases or decreases the level of expression of the RNA-binding protein HuR by more than three-fold in a sample of cancer cells contacted with the HuR-modulating agent compared to control sample of cancer cells not contacted with the HuR-modulating agent.

28. The composition of claim 27, wherein the level of expression of HuR is increased.

29. The composition of claim 27, wherein the level of expression of HuR is decreased.

30. The composition of claim 26, wherein the HuR modulating agent treats an HuR-mediated disease.

31. The composition of claim 30, wherein said HuR-mediated disease is cancer.

32. The composition of claim 31, wherein the cancer is breast cancer.

33. The composition of claim 32, wherein the cancer cells are estrogen receptor negative breast cancer cells.

34. The composition of claim 26, wherein the HuR-modulating agent comprises a single- or double-stranded nucleic acid comprising an HuR gene, or fragment thereof, operably-linked to a promoter active in cancer cells.

35. The composition of claim 34, wherein said nucleic acid is single-stranded.

36. The composition of claim 35, wherein said nucleic acid is single-stranded RNA.

37. The composition of claim 36, wherein said single-stranded RNA is packaged in a virus.

38. The composition of claim 37, wherein said virus is a retrovirus.

39. The composition of claim 38, wherein said retrovirus is a lentivirus.

40. The composition of claim 35, wherein said nucleic acid is double-stranded.

41. The composition of claim 40, wherein said nucleic acid is double-stranded DNA.

42. The composition of claim 41, wherein said double-stranded DNA is linear.

43. The composition of claim 42, wherein said linear double-stranded DNA is packaged in a virus.

44. The composition of claim 40, wherein said double-stranded DNA is circular.

45. The composition of claim 44, wherein said circular double-stranded DNA is a plasmid.

46. The composition of claim 44, wherein said circular double-stranded DNA is a packaged in a virus.

47. The composition of claim 34, wherein said HuR gene, or fragment thereof, encodes an HuR polypeptide, or a fragment or variant thereof, capable of binding to mRNAs encoded by one or more genes involved in angiogenesis or metastasis.

48. The composition of claim 47, wherein the level of expression of the HuR polypeptide, or a fragment of variant thereof, is increased in the cancer cells.

49. The composition of claim 47, wherein said HuR gene, or fragment thereof, is operably-linked to the promoter active in cancer cells in an anti-sense direction.

50. The composition of claim 49, wherein the level of expression of HuR is decreased in the cancer cells.
